(19) United States
(12) Patent Application Publication
(10) Pub. No.: US 2018/0125702 A1
(43) Pub. Date: May 10, 2018

(54) MEDICAL COOLING DEVICE AND METHOD FOR COOLING INFUSION FLUIDS

(71) Applicant: SEIRATHERM GMBH, Herzogenaurach (DE)

(72) Inventors: Matthias ROTH, Giggenhausen (DE); Thomas REICHTHALHAMMER, Unterschleißheim (DE)

(73) Assignee: seiratherm GmbH, Herzogenaurach (DE)

(21) Appl. No.: 15/564,350

(22) PCT Filed: Apr. 14, 2016
(86) PCT No.: PCT/EP2016/025039

§ 371 Date: Oct. 4, 2017

(30) Foreign Application Priority Data
Apr. 16, 2015 (EP) EP15001115.3

(51) Int. Cl. A61F 7/00; A61M 19/00

(52) U.S. Cl. CPC A61F 7/0085; A61F 7/007; A61M 19/00; A61M 2205/3673; A61F 2007/0076; A61F 2007/0093; A61M 2205/366; A61F 2007/0056

(57) ABSTRACT

The present invention relates to a medical cooling device for cooling at least one infusion fluid(s) comprising at least one Peltier element (10) having a cooling side (11) and a heating side (12) and being adapted and/or arranged to provide a first cooling power, at least one cooling duct (2) which is provided in, at and/or in the vicinity of the cooling side (11) of the Peltier element (10), the cooling duct (2) having an input port (21) where the infusion fluid enters and/or approaches the cooling side (11) of the Peltier element (10) or the vicinity thereof and an output port (22) where the infusion fluid exists or departs the cooling side (11) of the Peltier element (21) or the vicinity thereof and at least one further cooling stage (30) being adapted and/or arranged to generate a second cooling power which is adapted and/or arranged for cooling the heating side (11) of the Peltier element (10). The second cooling unit (30) has a second cooling power that is larger than the first cooling power of the Peltier element (10). The present invention also relates to a respective method.

### FIELD

The invention is directed to a hypothermia and a medical cooling device and method.

### BACKGROUND

Hypothermia is usually called a condition in which the body's core temperature drops below that required for normal metabolism and body functions. This is generally considered to be less than 35.0° C. (95.0° F.). Characteristic symptoms depend on the temperature. Targeted temperature management (TTM) previously known as therapeutic hypothermia or protective hypothermia is active treatment that tries to achieve and maintain a specific body temperature in a person for a specific duration of time in an effort to improve health outcomes. This is done in an attempt to reduce the risk of tissue injury from lack of blood flow. Periods of poor blood flow may be due to cardiac arrest or the blockage of an artery by a clot such as may occur in stroke. Targeted temperature management improves survival and brain function following resuscitation from cardiac arrest. Evidence supports its use following certain types of cardiac arrest in which an individual does not regain consciousness. Targeted temperature management following traumatic brain injury has shown mixed results with some studies showing benefits in survival and brain function while other show no clear benefit. While associated with some complications, these are generally mild. Targeted temperature management can advantageously prevent brain injury by several methods including decreasing the brain's oxygen demand, reducing the production of neurotransmitters like glutamate, as well as reducing free radicals that might damage the brain. The lowering of body temperature may be accomplished by many means including the use of cooling blankets, cooling helmets, cooling catheters, ice packs and ice water lavage.

Medical events that targeted temperature management may effectively treat fall into five primary categories: neonatal encephalopathy, cardiac arrest, ischemic stroke, traumatic brain or spinal cord injury without fever, and neurogenic fever following brain trauma.

According to EP 2010739239 A a hypothermia system comprises a fluid reservoir, a heat exchanger assembly, a catheter in fluid communication with the fluid reservoir, and a pump system configured to infuse hypothermic fluid into a patient cavity and extract hypothermic fluid from the patient cavity. The hypothermia system can infuse and extract fluid automatically from the patient cavity. In one embodiment, the patient cavity is a peritoneal cavity. A safe access device to gain access to the patient cavity is also provided. This, however, provides a rather voluminous system and makes it necessary to access a patient's cavity with a number of risks.

US 1967612849 A discloses a device for varying blood temperature comprising a Peltier block having a warm side and a cold side. A flow-through heat exchanger is connected to one of said sides of said Peltier block in thermal conduction there with and electrically insulated therefrom. The heat exchanger has a flow-through space traversed by a flow of blood, and said space extend band-shaped over substantially. The entire area of the Peltier block is on one of the sides thereof and has a width many times greater than the thickness thereof. A blood inlet to and outlet-from said flow through space is located at extremities thereof. The space is defined by smoothly polished surfaces and has fan-shaped transition portions respectively flaring from said inlet and narrowing to said outlet. The flow-through space has corners that are all rounded in outline. The fan-shaped portions are defined by lateral surface at the flaring sides thereof having a given degree of inclination cooperating with said rounded corners for avoiding stagnation of blood flowing through said space.

US 2006551235 A provides a system for chemo-hyperthermia treatment. The chemo-hyperthermia treatment system comprises a reservoir for storing fluid, a heating/cooling system coupled to the reservoir so that the fluid can be transferred from the reservoir to the heating system, wherein the heating/cooling system comprises a heating/cooling exchange module having a channel within which the fluid can flow. Moreover, a plurality of Peltier modules are coupled to the heating/cooling module, wherein the plurality of Peltier modules heat up the fluid flowing through the channel. In the cooling mode, the plurality of Peltier modules cool the fluid flowing through the channel. Further, a pumping means is coupled to the heating/cooling system, wherein the pumping means pump the perfusion fluid from the reservoir to the heating/cooling system, thereby allowing the heating/cooling system to change the temperature of the fluid; at least one inflow catheter coupled to the pumping means, wherein the at least one inflow catheter delivers the heated/cooled fluid to an object and at least one outflow catheter coupled to the reservoir. The at least one outflow catheter drains the fluid from the object to the reservoir.

### SUMMARY

The problem underlying the present invention is to provide an improved or ameliorated medical cooling device and/or medical cooling method for one or more infusion fluid(s).

The problem can be solved by the subject matter of the present invention exemplified by the description and the claims.

According to the present invention the cooling can be performed by a particular arrangement of an infusion tube and one or more particularly adapted Peltier elements and further cyclic refrigeration that can ensure a faster, quicker and/or more individualized temperature control of infusion fluids.

In particular, a medical cooling device for cooling an infusion fluid can comprise at least one Peltier element having a cooling side and a heating side and being adapted and/or arranged to provide a first cooling power. Further, at least one cooling duct can be provided in, at and/or in the vicinity of the cooling side of the Peltier element. The Peltier element is generally of known structure. The Wikipedia explanation httplide.wikipedia.org/wiki/Peltier-Element publicly available on Apr. 6, 2015 is herein incorporated by reference. The cooling duct and finally the infusion fluid(s) can be cooled by the Peltier element. The cooling duct can also be a duct hosting another duct or other ducts or a duct cartridge in its interior. In this manner the interior duct can be easily replaced in order to keep the system sterile or semi-sterile. Also a second or more Peltier element(s) can be arranged along the cooling duct in order to have a longer length of cooling duct or several parallel ducts being exposed to the cooling effect of the Peltier element(s). Moreover, the cooling duct comprises an input port where the infusion fluid enters and/or approaches the cooling side of the Peltier element or the vicinity thereof and an output port where the infusion fluid exists or departs the cooling side of the Peltier element or the vicinity thereof. As mentioned before, this also comprises a duct-in-the-duct or cartridge arrangement. Further, at least one further or second cooling stage or second cooling unit is adapted and/or arranged to generate a second cooling power which is adapted and/or arranged for cooling the heating side of the Peltier element. The second cooling stage has a second cooling power that can be larger than the first cooling power of the Peltier element.

The infusion fluid can be further infused into a patient. Alternatively, it can be expelled into a container in order to provide cooled infusion fluid and/or in order to test the device and/or method according to the present invention.

The first cooling power of the Peltier element and the second cooling power of the second cooling stage can be adapted and arranged to cool the infusion fluid by at least 11° C., preferably by at least 14° C., more preferably by at least 16° C., even more preferably by at least 17° C., most preferably by at least 18° C. In the latter case an infusion fluid having a room temperature of 21° C. can be cooled down to up to 4° C. or less which has been turned out to be a well suited temperature in order to bring down the temperature of a patient or of parts of his body, such as the brain, down to 35° C., even 32° C. or cooler as soon as possible without harming the consistence of the infusion fluid. Anyhow, other even higher temperatures than 4° C. of the infusion fluid may be suited to enable hypothermia, depending on the needs.

The first cooling power of the Peltier element and the second cooling power of the second cooling stage of the cooling element can be adapted and/or arranged to cool the infusion fluid with a flow rate of at least 1.5 ltr./h, preferably by at least 1.75 ltr/h, more preferably by at last 2 ltr/h, more preferably by at least 2.5 ltr/h, more preferably by at least 3.0 ltr./h, more preferably by at least 4.0 ltr./h, and more preferably by at least 4.5 ltr./h.

A ratio of the second cooling power to the first cooling can be at least 2:1, preferably at least 2.5:1, more preferably at least 3:1, even more preferably at least 3.5:1 and most preferably at least 4:1.

The second cooling stage can be a cyclic refrigeration unit. Such units are described, e.g., on http://de.wikipedia.org/wiki/Källmaschine available on Apr. 6, 2015 and incorporated herein by reference. The second cooling stage can be a vapor compression cycle unit. Any other cyclic refrigeration can also be used, such as gas cycle refrigeration and/or vapor-absorption refrigeration. Alternatively and/or additionally a vapor absorption cycle, a gas cycle, an air cycle or a magnetic refrigeration can be used.

A cooling body can form itself or can host the cooling duct. The cooling duct can be provided in, within or adjacent to the cooling side of the Peltier element. The infusion fluid flowing or being drawn through the cooling duct can thus be cooled. The cooling body can be cup-shaped in order to cover a front face and side faces of the cooling body and/or of the Peltier element. Thus, the cooling effect of the entire free or open surface of the cooling side of the Peltier element can be used. However, depending on the design, the cooling effect of the face of the cooling side of the Peltier element can be sufficient.

Examples of materials for the cooling body or a cooling block are aluminum, copper, alloys thereof, ceramic and/or carbon materials. Alternatively, the cooling body or the cooling clock can be made of or comprise plastics materials using integrated powders, fibers and/or filaments preferably in a polymer matrix. Such powders, fibers and/or filaments preferably comprise metals or alloys thereof or can be made of these. The matrices can be made of or contain thermoplastics such as polyolefin and particularly polypropylene. In case of particular thermal stresses polyamides and/or polyphenylsulfides.

The cooling body or the cooling block can be brought into shape by molding and/or forming and/or pressing and to allow a close fit to the outer contours of the elements the cooling bodies or Peltiers are attached to. Moreover a better temperature flow along certain profiles can be obtained by modifying the internal structure of these elements. Alternatively or additionally such elements can be glued or otherwise attached with water containing materials further supporting the heat flow.

The cooling body is adapted and/or arranged in order to allow the cooling duct or a part thereof to be placed into the cooling body. The cooling body can be formed by two body parts that are hinged or assembled onto each other in order to be opened in order to allow the cooling duct or a part thereof to be placed into the cooling body, both body parts having respective cavities that allow both body parts to snugly encase the cooling duct when being closed.

Further, at least one thermally insulating layer can be further provided for thermally insulating the cooling body and/or the cooling side of the Peltier element. This may prevent or minimize the warming of the cooling side and the further attached elements by the ambient temperature or an even elevated temperature within the device. The insulation layer(s) can be placed around the cooling side of the Peltier element and/or the cooling body. Such temperature insulation layer(s) can be made of or contain the following or parts thereof: wood, rubber, foams, mineral wools, glass wools, plastics and/or natural damping materials, cork, foams, polystyrol, polyurethane and/or vacuum insulating plates.

The insulating layer can cup-shaped and enclose open sides of the cooling body of the and /or of the cooling side of the Peltier element. It can also be individually shaped, molded or cast to the shapes needed.

A source of infusion fluid can be attached to the cooling duct. The infusion fluid can be any among known infusion fluids such as blood/blood derivates and fluid infusion systems and/or an infusion system for infusing, e.g., saline or other balanced fluids like ringer's solution. Also the kind, shape, material and volume can vary.

The second cooling stage can comprise at least one cyclic refrigeration unit, as mentioned before but not excluding other alternatives, and at least one second thermally and/or electrically insulating layer can be placed within the second cooling stage so as to thermally insulate the Peltier element and the member(s) of the second cooling stage, such as an evaporator directly cooling the heating side of the Peltier element from the rest of the second cooling stage. This has two advantageous (but not necessary) effects: first, the temperature of the heating side of the peltier element is not exposed to the remaining parts of the second cooling stage and will not lead to influences in its cooling power (größter Vorteil des zweiten Kühlkreislaufs: Peltierkühlung funktioniert immer relativ zur Umgebungstemperatur bzw. zur Temperatur der Warmseite. Wird erst später beschrieben, sollte meiner Meinung aber hier bei den Vorteilen schon erwähnt werden); second, the high voltage part of the medical device is also electrically insulated which can be another safety measure towards a patient potentially connected to the infusion fluid.

The second cooling stage can comprise a compressor, a condenser and a throttle. However, any other elements and/or arrangement can be used, as stated before and below.

Moreover, a controller (not shown) can be provided and adapted and/or arranged to control the Peltier element and the second cooling stage in a manner that both are activated for delivering a high cooling power to the infusion fluid and only the Peltier element is activated for delivering a lower cooling power and/or stable cooling power to the infusion fluid. Other modes of operation may also be controlled by the controller. The operation of the different elements may be a non-feedback or at least one feedback controlled loop(s). The respective elements can be sensors measuring flow rates of the infusion fluid and/or temperatures of the infusion fluid upstream and/or downstream the cooling duct(s). Moreover, temperature sensors may be provided for measuring the temperature in the or at the cooling block, the cooling side of the peltier, the heating side of the peltier or at distinct locations of the second cooling stage. Moreover, a memory with one or more models or look-up tables for controlling the temperature of the infusion fluid leaving the cooling duct may be provided. Depending on the different measures or estimations the different components of the medical device can be operated or controlled.

The present invention also comprises a method of operation, particularly of any one of the before or below described device, with the step of providing at least one Peltier element with a cooling side and a heating side and delivering a first cooling power. At least one cooling duct in, at and/or in the vicinity of the cooling side of the Peltier element can be provided. Moreover, the cooling duct can be provided with an input port for allowing the infusion fluid to enter or approach the cooling side of the Peltier element or the vicinity thereof and with an output port for allowing the infusion fluid to exit or depart the cooling side of the Peltier element or the vicinity thereof. Even further a second cooling stage or cooling unit is provided generating a second cooling power and cooling the heating side of the Peltier element. The second cooling power can be larger than the first cooling power. This may bring the Peltier element(s) to a higher and more rapid cooling power or performance.

Moreover a cooling method is embraced with a cooling device comprising the steps of arranging a first cooling power and a second cooling power different from the first cooling power in series and applying the first cooling power to the cooling fluid, applying first the first and the second cooling powers and subsequently just the first cooling power to cool the infusion fluid. In series means that the second cooling stage of any of the described natures can cool the heating side of the Peltier element.

All aspects of the present invention are adjusted to operate or be operated without a patient. According to one aspect of the present invention the infusion fluid can be collected by a container or can be infused into a patient.

The present invention can preferably provide the advantage to generate faster and further preferably more precisely adjusted or positively controlled temperatures of the infusion fluid. Thus, more individualized and a better adjusted flow of one or more infusion fluid(s) can be realized or a patient who can be treated better according to the needs detected in real time or close to real time.

These and other features of the present teachings are set forth herein.

### DRAWINGS

The skilled artesian will understand the drawings, described below, are for illustration purposes only. The drawings are not intended to limit the scope of the present teaching in any way.

FIG. 1 is a principal sketch of a medical cooling device showing a front perspective onto the device and how elements thereof can be arranged;

FIG. 2 is a principle top view sketch into the medical cooling device how some of the further elements thereof can be functionally arranged;

FIG. 3 is a principal sketch of cooling Peltier elements and how they can be arranged;

FIG. 4 exemplifies a typical and principal cooling effect of a Peltier element to an infusion fluid over time;

FIG. 5 exemplifies a typical and principal cooling effect of a cyclic refrigeration element to an infusion fluid over time;

FIG. 6 exemplifies a typical and principal cooling effect of a combined Peltier element and cyclic refrigeration element according to an aspect of the present invention for an infusion fluid over time.

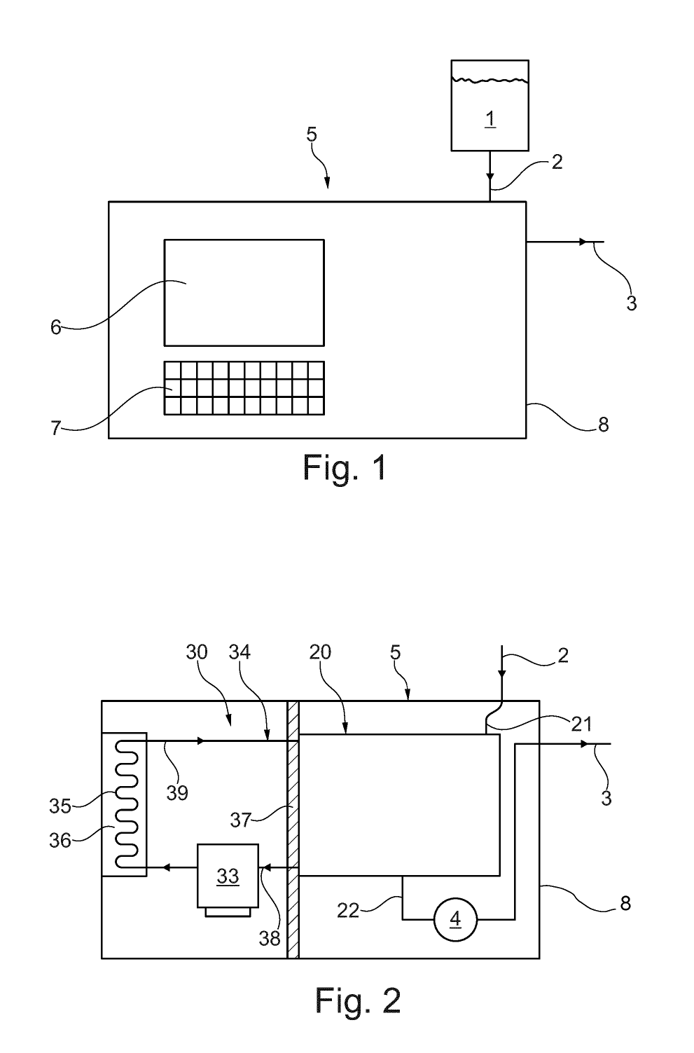

FIG. 1 exemplifies how elements according to the present can be (but must not be) arranged. A source 1 with an infusion fluid is shown which can have the known structure and form of known infusion bags. The source 1 can also comprise more than one containers of the same or different shapes and/or with the same or different infusion fluids. They can be made of a translucent of transparent plastic material or any other material and can have the shape of a bag or any other shape.

A duct 2 delivers the fluid out of the source 1. The duct 2 can also contain more than one ducts, can be rigid, semi-rigid or flexible. Further they can be made of any material. Commonly transparent materials are used. Other common elements like a drip dosing device or valve-like hand-actuated restricting element are not shown but can be contained. The same holds true for other standard elements or components used for infusion purposes.

FIG. 1 also depicts schematically a medical cooling device 5. The medical cooling device can be integrated into one housing 8 but can also be composed of two or more modular elements. The shape can also be different to the one shown. The medical cooling device 5 can be made of any suitable material. Further, it can comprise any kind of a display 6, such as an LED display. The display 6 can be a touch panel display, such as a capacitive display. Alternatively or additionally a keypad 7 can be provided. Anyhow, one or both, can also be omitted. This may be suitable in case there is a central controlling device close by and/or distant in a central controlling station (not shown but comprised by the present invention).

FIG. 2 shows some of the elements for cooling the infusion fluid(s) in the medical cooling device 5. As can be seen the duct 2 enters the medical cooling device. While it is shown in an upper and side portion of the medical cooling device 5 any other location can be used whatever is suitable. The duct further enters a first cooling stage 20 which will be explained later in more detail. It is also possible to provide a coupling 21 (not shown in more detail) where the duct 2 is coupled to the further duct for leading the infusion fluid to and through the cooling stage 20. At the bottom the second duct 3 leaves the first cooling stage 20. A second coupling 22 can be provided (not shown in more detail). It can also be provided at the housing 8 of the medical cooling device 5.

A pump 4 can be provided if suitable. It will transport the fluid through duct 3 to a collection container (not shown) or a patient (not shown). The pump 4 can also be adapted and develop a negative pressure to pull the fluid through duct 2 and the first cooling stage 20. Particularly in order to control the amount of volume per time or the flow rate the pump 4 can be provid. It can be any type of pump used in medical devices, such as a dosing pump, a peristaltic pump, a piston pump, a turning pump etc. The pump 4 is controlled by a controller (not shown) and can also be feed-back controlled by a flow meter and the controller (both not shown).

FIG. 2 also shows the second cooling stage or second cooling unit 30. Cooling device according to any one of the preceding claims wherein the second cooling stage 30 is a cyclic refrigeration unit 30 in which a refrigerant undergoes phase changes. This type of refrigeration is highly effective. The second cooling stage 30 can be a vapor compression cycle unit. In FIG. 2 a compressor 33 is shown. A duct 38 can convey a refrigerant and compress it. In a condenser 35, 36 comprising a condenser structure 36 the refrigerant can be condensed. The refrigerant can be guided through the condenser in one or more ducts 36 in a wound or meander fashion. In FIG. 2 the meander is shown to meander vertically for demonstration purposes. It can also meander in a horizontal fashion. The refrigerant is further drawn out of the condenser 36 in the duct 39 through a throttle 34. The refrigerant will then be conveyed to a cooling part or an evaporator that will be explained later. A thermally and/or electrically insulating material 37 forming a separation or wall can also be provided in the medical cooling device thermally isolating the evaporator from the remaining parts of the second cooling stage.

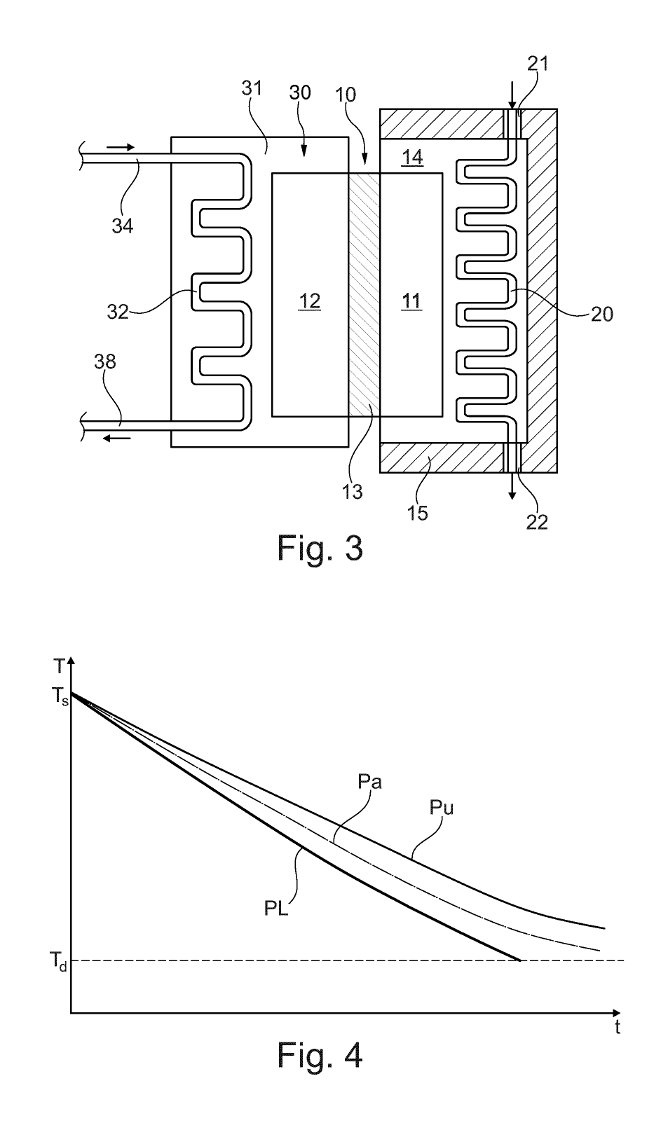

FIG. 3 shows the first cooling stage 20 and part of the second cooling stage in more detail. In continuation to the above, the evaporator 31, 32 of the second cooling stage is shown in more detail. Within the evaporator structure one or more evaporator ducts 32 are provided that lead the refrigerant through the evaporator structure in a wound or meander fashion.

The first cooling stage 20 comprises a Peltier element 10. The Peltier element comprises a heating side 12. As is apparent, the heating side 12 of the Peltier element can be cooled by the second cooling stage 30. In the (non-exclusive) embodiment shown, the heating side 12 of the Peltier element 10 is embraced by the cooling parts 31, 32 of the second cooling stage. The embracement can be cup-shaped or C-shaped as shown in the two-dimensional drawing. Alternatively it can be coupled just to the face of the heating side 12 or parts thereof and/or it can be glued by water-containing glue in order to improve heat transmission.

Peltier element 10 also comprises a cooling side 11 that is separated by an insulating layer 13 from the heating side 12. According to the present invention this constitutes a preferred advantage as the high voltage parts of the medical cooling device are electrically separated or insulated by the insulating layer 13 towards the infusion fluid or any parts being connected or adjacent the infusion fluid.

The Peltier element 10 is a thermoelectric cooler or TEC using the Peltier effect to create a heat flux between the junction of two different types of materials. The Peltier cooler is a solid-state active heat pump which transfers heat from the cooling side 11 to the heating side 12 with the consumption of electrical energy.

In FIG. 3 the cooling side 11 is attached or adjacent a heat exchanger 14, 20. Preferably the heat exchanger comprises a block 14 made of or comprising a material with good or excellent heat conducting properties. Some materials are mentioned above.

Throughout the block 14 a heat exchanging or cooling duct 20 is provided. This heat cooling duct 20 can have more than one lines of flow, such as two or more lines of flow separating at an entrance port 21 and merging at an exit port 22. Additionally, two and more cooling ducts 20 can be provided for allowing two or more infusion fluids to be cooled.

The cooling side 11 of the Peltier element conducts the low temperature to the block 14. The infusion fluid running or being forced through the cooling duct 20 is cooled down during its flow. There may be further means disturbing the laminar flow for fluid through duct 20 and creating turbulent flow in order to also bring as many fluid molecules as possible in contact or in the vicinity of walls of the cooling duct 20. A cooling duct 20 or a plurality thereof can be placed or formed in the cooling side 11 or adjacent the cooling side 11 of one or more Peltier elements 10.

According to one aspect of the invention the cooling duct(s) 20 can have a meander shape, sinuous shape or any other winding shape in order to prolong the length of the cooling duct 20 in, at and/or adjacent the cooling side 12 of the Peltier element. Additionally or alternatively, the cooling duct 20 can be composes of two or more sections with different shapes and/or different properties. This enables a longer exposure of an infusion fluid flowing in the cooling duct 20 to the cold temperature of the cooling duct 20 and an improved cooling rate or speed of the infusion fluid. Additionally or alternatively one or more of the cooling ducts 20 or portions thereof can be provided with internal ribs, ridges etc. in order to enlarge to surface of the cooling duct 20 exposed to the infusion fluid and/or to interrupt a laminar flow and/or to cause a non-laminar flow of the infusion fluid flowing through the cooling duct 20, as also mentioned before.

In order to keep the cooling side as cool as possible and to prevent warming by any ambient air in or around the medical cooling device a thermally insulating material 15 can surround the face of the cooling side 11, parts thereof or even the side with a cup-shaped structure.

FIG. 3 exemplifies one aspect of the present invention. A source 1 of infusion fluid can be hung up or placed in any manner in order to deliver the infusion fluid to a patient (not shown) or a container (not shown) of any kind and for testing or other purposes. The infusion fluid is delivered by a duct 2 or pipe 2. The duct 2 merges, is connected to or is unitary with a cooling duct 20.

A Peltier element 10 is shown with a cooling side 11 and a heating side 12. Both sides are separated by an electrical insulation layer 13. The Peltier element 10 can be of standard type with appropriate sizes, forms and/or cooling properties or can be customized in size, shape and/or cooling properties in order to improve cooling speeds, cooling capacities, speeds of temperatures or temperature changes to be adjusted etc.

According to a further preferred aspect of the present invention another cooling body 14 is formed adjacent the cooling side 12 of the Peltier element 10. The cooling body 14 is attached to the cooling side 11 of the Peltier element 10 in order to improve the temperature flow as quickly as possible and/or to minimize any losses to the environment. In order to realize this the cooling body is firmly attached to the cooling side 11 of the Peltier element 10 with low losses in temperature flow and/or the cooling body 14 is made of a material with low temperature flow resistance and/or high temperature conduction.

Within the cooling body 14 the cooling duct(s) 20 can be placed or integrally formed with the cooling body 14. The cooling body 14 can be taken apart or opened by two half bodies 14a, 14b to be secured together to form the cooling body 14. According to a preferred aspect of the present invention the two half bodies 14a. 14b are hinged together and can be secured by any known means. This assists in keeping the cooling duct 20 and particularly its inner side sterile. The cooling duct 20 can be put in a temper evident and sterile package or blister package to be opened before the cooling duct 20 is placed into the cooling body 14.

According to another aspect of the present invention the cooling duct 20 can be integrally formed in the cooling body 14. This can be done by milling a part thereof in one half of the cooling body 14, the other part in the other half of the cooling body 14 and both half bodies of the cooling duct then be firmly attached to each other. This can be done by screw bolts connecting the two half bodies.

The heating side 12 of the Peltier element 10 is further cooled by a further second cooling stage or unit 30. The term “stage” or “unit” shall also comprise any element or assembly of a plurality of elements cooperating in a cyclic cooling or cyclic refrigeration. The second cooling power or refrigeration power and/or temperature range of the second cooling unit (30) is larger than the first cooling power or refrigeration power and/or temperature range of the Peltier element (10).

The provision of a Peltier element 10 with a separation layer 13 as well as a second cooling unit 30 makes it possible to electrically separate the electrical energy sources driving the Peltier element 10 and the second cooling unit 30. Moreover, the two or more step approach is able to cool more effective, faster and in real time or almost in real time the infusion fluid to be applied.

This is able to quickly, precisely and easily generate cold temperatures in or at the heating element of the Peltier element 10.

### COMPARATIVE EXAMPLES

The present invention and aspects thereof have been compared to existing technologies and/or suggestions according to the prior art.

FIG. 4 exemplifies a typical and principal cooling effect of a Peltier element to an infusion fluid over time. As can be seen by the line Pa (actual Peltier behavior) it takes a rather long time or larger Peltier elements to cool down an infusion fluid from a starting temperature Ts to a desired temperature Td. Without knowing the actual behavior of a Peltier element Pa can be estimated at a starting point or later to lie between a lower limit or expected minimum PI and an upper limit or expected maximum Pu. Compared with a typical cyclic refrigeration element the cooling power is lower but the control and/or behavior show narrow tolerances.

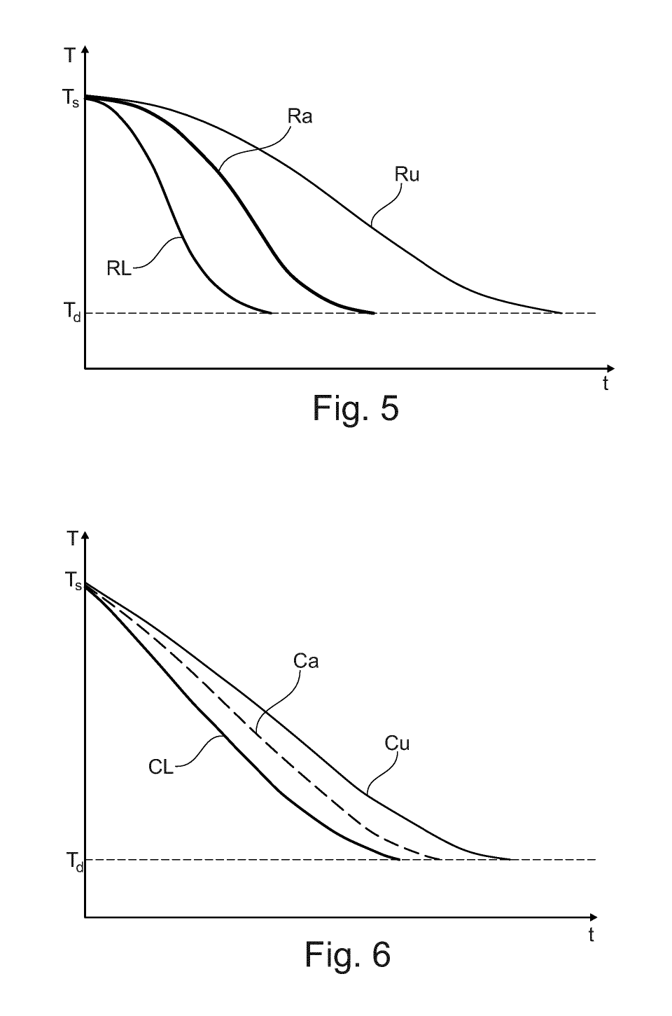

FIG. 5 exemplifies a typical and principal cooling effect of a cyclic refrigeration element to an infusion fluid over time. The actual behavior Ra of the cyclic refrigeration element allows a quicker cooling down of an infusion fluid. However, at the beginning or close to the starting temperature Ts there is a retention time and the control or estimation usually requires a broader tolerance between a lower limit or expected minimum Rl and an upper limit or expected maximum Ru.

FIG. 6 exemplifies a typical and principal cooling effect of a combined Peltier element and cyclic refrigeration element according to an aspect of the present invention for an infusion fluid over time. As can be seen, the actual behavior of the combined arrangement shown with line Ca avoids the retention time of a cyclic refrigeration element, results in a faster cooling behavior of the infusion fluid and a better behavior in a more narrow tolerance field between a lower limit or expected minimum Cl and an upper limit or expected maximum Co. This can facilitate the control of the combined arrangement and can result in a considerably faster and better predictable cooling of the infusion fluid.

Thus, it has been found that the present invention and aspects thereof can deliver a faster and further preferably more precisely adjusted or positively controlled temperatures of the infusion fluid. Thus, more individualized and a better adjusted flow of infusion fluids can be realized or a patient can be treated more according to the needs detected in real time or close to real time.

While the disclosure has been illustrated and described in detail in the drawings and foregoing description, such illustration and description are to be considered illustrative or exemplary and non-restrictive; the disclosure is thus not limited to the disclosed embodiments. Variations to the disclosed embodiments can be understood and effected by those skilled in the art and practicing the claimed disclosure, from a study of the drawings, the disclosure, and the appended claims. In the claims, the word “comprising” does not exclude other elements or steps, and the indefinite article “a” or “an” does not exclude a plurality. A single processor or other unit may fulfill the functions of several items recited in the claims. The mere fact that certain measures are recited in mutually different dependent claims does not indicate that a combination of these measures cannot be used to fulfill aspects of the present invention. The present technology is also understood to encompass the exact terms, features, numerical values or ranges etc., if in here such terms, features, numerical values or ranges etc. are referred to in connection with terms such as “about, ca., substantially, generally, at least” etc. In other words, “about 3” shall also comprise “3” or “substantially perpendicular shall also comprise “perpendicular. Any reference signs in the claims should not be considered as limiting the scope.

In the claims, the term “comprises/comprising” does not exclude the presence of other elements or steps. Furthermore, although individually listed, a plurality of means, elements or method steps may be implemented. Additionally, although individual features may be included in different claims, these may possibly advantageously be combined, and the inclusion in different claims does not imply that a combination of features is not feasible and/or advantageous. In addition, singular references do not exclude a plurality.

1. Medical cooling device for cooling an infusion fluid, comprising
a. a Peltier element having a cooling side and a heating side and being adapted and/or arranged to provide a first cooling power,
b. a cooling duct which is provided in, at and/or in the vicinity of the cooling side of the Peltier element,
c. the cooling duct having an input port where the infusion fluid enters and/or approaches the cooling side of the Peltier element or the vicinity thereof and an output port where the infusion fluid exists or departs the cooling side of the Peltier element or the vicinity thereof, d. a second cooling stage being adapted to generate a second cooling power which is adapted and/or arranged for cooling the heating side of the Peltier element,
e. wherein the second cooling power that is larger than the first cooling power.

2. Cooling device according to claim 1, wherein the first cooling power of the Peltier element and the second cooling power of the second cooling stage are adapted and arranged to cool the infusion fluid by at least 11° C.

3. Cooling device according to claim 2, wherein the first cooling power of the Peltier element and the second cooling power of the second cooling stage of the cooling element are adapted and/or arranged to cool the infusion fluid with a flow rate of at least 1.5 ltr/h.

4. Cooling device according to claim 1, wherein the ratio of the second cooling power to the first cooling power is at least 2:1.

5. Cooling device according to claim 1, wherein the second cooling stage is a cyclic refrigeration unit.

6. Cooling device according to claim 1, wherein the second cooling stage is a vapor compression cycle unit.

7. Cooling device according to claim 1, wherein further comprising a cooling body for forming or hosting a cooling duct is further provided in, within or adjacent to the cooling side of the Peltier element.

8. Cooling device according to claim 7, wherein the cooling body is cup-shaped surround a front face and side faces of the cooling body and/or of the Peltier element.

9. Cooling device according to claim 8, wherein the cooling body is adapted and/or arranged to allow the cooling duct or a part thereof to be placed into the cooling body.

10. Cooling device according to claim 8, wherein the cooling body is formed by two body parts being hinged or assembled onto each other in order to be opened to allow the cooling duct or a part thereof to be placed into the cooling body, both body parts having respective cavities that allow both body parts to snugly encase the cooling duct when being closed.

11. Cooling device according to claim 7, further comprising a thermally insulating layer for thermally insulating the cooling body and/or the cooling side of the Peltier element.

12. Cooling device according to claim 11, wherein the insulating layer is cup-shaped and encloses open sides of the cooling body and /or of the cooling side of the Peltier element.

13. Cooling device according to claim 7, with a source of the infusion fluid being attached to the cooling duct.

14. Cooling device according to claim 11, wherein the second cooling stage comprises at least one cyclic refrigeration unit and wherein a second thermally and/or electrically insulating layer is placed within the second cooling stage so as to thermally insulate the Peltier element and an evaporator of the second cooling stage directly cooling the heating side of the Peltier element from the rest of the second cooling stage.

15. Cooling device according to claim 1, wherein the second cooling stage comprises a compressor, a condenser and a throttle.

16. Cooling device according to claim 1, further comprising a controller being adapted and/or arranged to control the Peltier element and the second cooling stage in a manner that both are activated for delivering a high cooling power to the infusion fluid and only the Peltier element is activated for delivering a lower cooling power and/or stable cooling power to the infusion fluid.

17. Method of cooling an infusion fluid with a cooling device, comprising: of:
a. providing a Peltier element with a cooling side and a heating side and providing a first cooling power,
b. providing a cooling duct in, at and/or in the vicinity of the cooling side of the Peltier element,
c. providing the cooling duct with an input port for allowing a cooling fluid to enter or approach the cooling side of the Peltier element or the vicinity thereof and with an output port for allowing the cooling fluid to exit or depart the cooling side of the Peltier element or the vicinity thereof,
d. providing a cooling stage and generating a second cooling power and cooling the heating side of the Peltier element,
e. wherein the second cooling power is larger than the first cooling power.

18. Method of cooling the infusion fluid according to claim 17, comprising: arranging the first cooling power and the second cooling power different from the first cooling power in series and applying the first cooling power to the cooling fluid, applying first the first and the second cooling powers and subsequently just the first cooling power to cool the infusion fluid.
